(19) United States
(12) Patent Application Publication
(10) Pub. No.: US 2002/0004593 A1
(43) Pub. Date: Jan. 10, 2002

(54) METHODS OF MAKING MODIFIED NUCLEOSIDES

(76) Inventors: THOMAS J. MEADE, ALTADENA, CA (US); CHANGJUN YU, PASADENA, CA (US)

Correspondence Address: ROBIN M SILVA, FLEHR HOHBACH TEST ALBRITTON & HERBERT, SUITE 3400 FOUR EMBARCADERO CENTER, SAN FRANCISCO, CA, 941114187

(21) Appl. No.: 09/192,167

(22) Filed: Nov. 13, 1998

(51) Int. Cl. C07H 19/00; C07H 21/00; C07H 21/02; C07H 21/04; C07H 19/04; C07H 19/16; C07H 19/22

(52) U.S. Cl. 536/25.32; 536/25.34; 536/26.6; 536/27.11

(57) ABSTRACT

The invention is directed to novel methods of making nucleosides modified with signalling moieties and polydentate ligands, particularly for use in chelating transition metal complexes to form signalling moieties such as electron transfer moieties and fluorophores.

### FIELD OF THE INVENTION

The invention is directed to novel methods of making nucleosides modified with signalling moieties and polydentate ligands, particularly for use in chelating transition metal complexes to form signalling moieties such as electron transfer moieties and fluorophores.

### BACKGROUND OF THE INVENTION

Due to the expanding field of nucleic acid detection, including both clinical and research applications, the labeling of nucleosides and nucleic acids with detectable signalling moieties is of great interest. These signalling moieties can be detected on the basis of electronic and electrochemical properties (see for example U.S. Pat. Nos. 5,591,578; 5,824,473; 5,770,369; 5,705,348 and 5,780,234 and PCT 97/20014); fluorescence (see Harnes and Higgins, Eds. Gene Probes 1 (IRL Press, New York, 1995); Mansfiled et al., Mol. Cell Probes 9:145 (1995); Kricka, Ed. Nonisotopic DNA Probe Techniques (Academic Press, San Diego, 1992); Tyagi et al., Nature Biotech. 14:303 (1996)); radioactivity (see Harnes, supra, and U.S. Pat. No. 4,707,352); electrochemiluminescence (see W090/05301; WO 92/14139; and U.S. Pat. Nos. 5,779,976 and 5,770,459), etc.

There is a large body of work directed to the attachment of signalling moieties to the base of nucleosides; see for example, U.S. Pat. Nos. 5,002,885; 5,476,928; 5,449,767; 4,711,955; 4,952,685; 5,175,269; 5,241,060; and 5,328,824, and related patents.

Chemical modifications on the ribose have been done; see U.S. Pat. No. 4,849,513;Sebesta et al., Tetrahedron 52:14385 (1996); McGee et al., J. Org. Chem. 61:779 (1996); Moffatt, Transformations of the Sugar Moiety of Nucleosides, pp71-164; Kirschenheuter et al., Tetrahedron Lett. 35:8517 (1994), and related materials.

However, there is a need for a simple and rapid method for the attachment of signalling moieties to nucleosides, for incorporation into nucleic acids.

### SUMMARY OF THE INVENTION

In accordance with the objects outlined above, the present invention provides methods for making a modified nucleoside comprising a covalently attached signalling moiety or signalling moiety precursor (i.e. polydentate ligand). The methods comprising adding an anhydro-nucleoside and a signalling moiety or signalling moiety precursor comprising a primary amine. The addition is done in the presence of an activation agent to form an activated anhydro-nucleoside. The activated anhydro-nucleoside is treated with a cyclization agent to form a cyclized intermediate. The cyclized intermediate is treated with a base to form the nucleoside modified with a signalling moiety or polydentate ligand.

### BRIEF DESCRIPTION OF THE DRAWINGS

FIG. 1 depicts the generic synthetic scheme of the methods of the invention for the production of a nucleoside modified with a signalling moiety or precursor. “A” in this case represents a signalling moiety or signalling moiety precursor (i.e. polydentate ligand). The scheme depicts the use of anhydrouracil as the anhydronucleoside, although as outlined herein others may be used. Similarly, the activation agent shown is carbonyldimidazole.

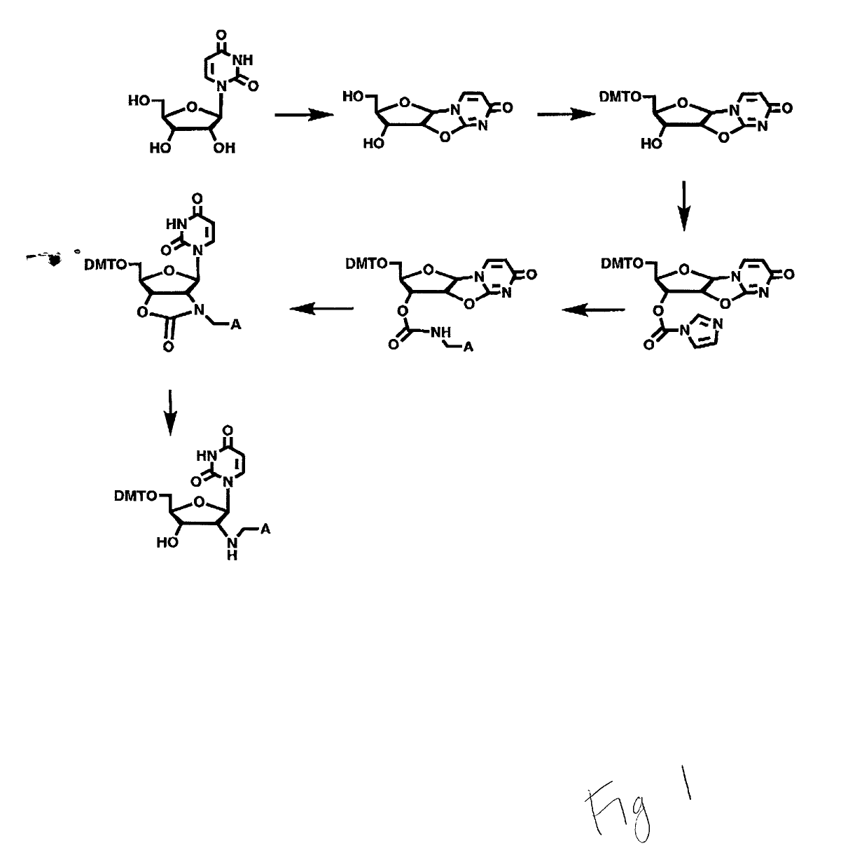

FIG. 2 depicts the synthesis of a nucleoside modified with a specific polydentate ligand, aminomethylpyridine.

FIG. 3 depicts the synthesis of a nucleoside modified with a ruthenium complex.

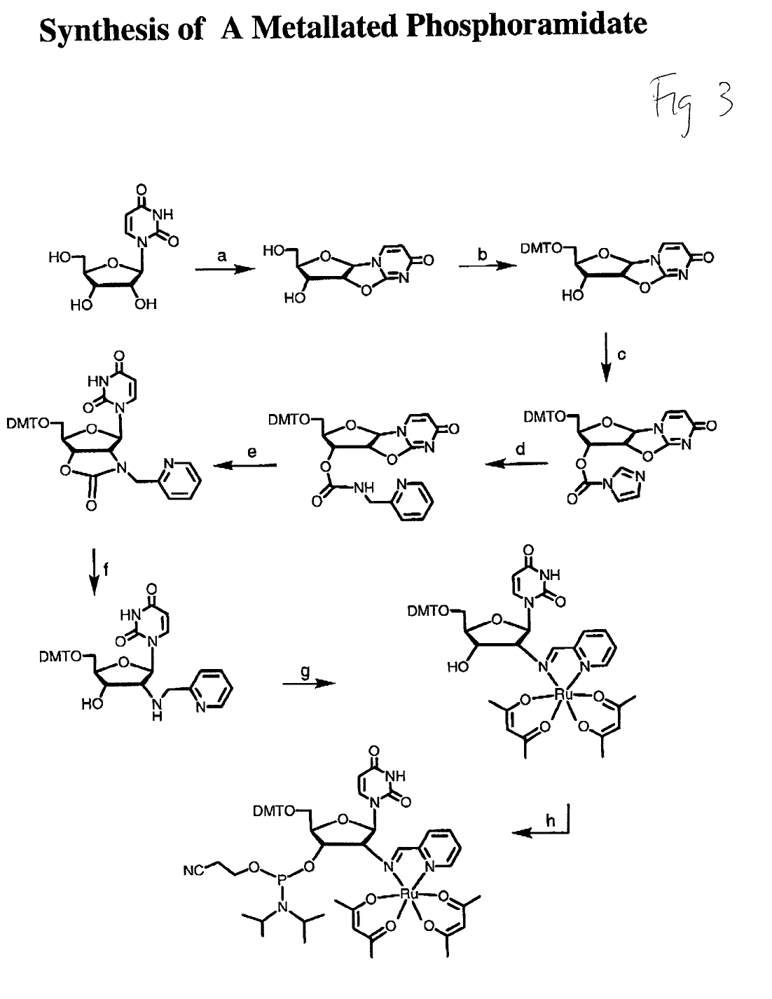

### DETAILED DESCRIPTION OF THE INVENTION

The present invention is directed to a rapid, simple synthesis of nucleosides covalently modified at the 2′ or 3′ position of the ribose with a signalling moiety or signalling moiety precursor. These modified nucleosides may then be incorporated into a growing nucleic acid to form a nucleic acid with a covalently attached signalling moiety or signalling moiety precursor.

Accordingly, the present invention provides methods for making modified nucleosides comprising a covalently attached signalling moiety or signalling moiety precursor. By “nucleoside” herein is meant a base attached to a ribose (furan). The base may be any base that can form an anhydro-structure, as defined below, including naturally occuring and non-naturally occuring bases. Suitable bases include, but are not limited to, uracil, thymine, cytosine and inosine, and base analogs such as xathanine, hypoxathanine, isocytosine, halogenated bases such as 5-halo-uracil (e.g. 5-bromo- or 5-iodo-uracil), etc. The ribose may be either ribose or ribose analogs such as the five membered carbon ring analogs, etc. Accordingly, as used herein, the term “nucleoside” includes nucleoside analogs. While the nucleic acids of the present invention will generally contain phosphodiester bonds, in some cases nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done for a number of reasons, including for example to increase the stability and half-life of such molecules in physiological environments.

As will be appreciated by those in the art, all of these nucleic acid analogs may find use in the present invention. In addition, mixtures of naturally occurring nucleic acids and analogs can be made; for example, at the site of signalling moiety attachment, an analog structure may be used. Alternatively, mixtures of different nucleic acid analogs, and mixtures of naturally occuring nucleic acids and analogs may be made.

As used herein, the term “nucleosides” may also include nucleotides, i.e. nucleosides with phosphate groups attached, and nucleotide analogs as outlined above. Thus, for example, having synthesized a modified nucleoside, a triphosphate nucleotide may be formed, or a phosphoramidite form of the nucleoside may be formed, for incorporation into a nucleic acid. “Nucleic acid” in this context means two or more nucleosides joined together, and can have any combination of natural and synthetic bases, including uracil, adenine, thymine, cytosine, guanine, inosine, xanthanine, hypoxanthanine, isocytosine, isoguanine, halogenated bases, etc.

By “modified nucleoside” herein is meant a nucleoside comprising a signalling moiety or signalling moiety precursor (i.e. a polydentate ligand) covalently attached to the 2′ or 3′ position of the ribose (or the equivalent position in a ribose analog). By “signalling moiety” herein is meant any moiety which can be used to detect the nucleoside or nucleic acid into which it is incorporated. As will be appreciated by those in the art, the characterization and identity of the signalling moiety will depend on the desired detection method. Thus, for example, signalling moieties comprising electron transfer moieties, optical dyes including fluorochromes and chromophores, chemiluminescent and electrochemiluminescent labels, magnetic resonance imaging (MRI) agents, enzymes, haptens and other binding ligands to which a labelled binding partner may be attached (e.g. digeoxigenin, biotin, antigens, nucleic acids, etc.), can all be attached to nucleosides using the methods of the present invention, as long as a derivative can be made that includes a primary amine that does not eliminate its signalling properties.

As outlined herein, the signalling moiety must contain a primary amine. By “primary amine” herein is meant an —NH₂ group. It should be understood that this term includes —NH₂ groups irrespective of the group to which the primary amine is attached; for example, as used herein, the nitrogen containing moieties of R—CO—NH₂ and R—O—NH₂ are both considered primary amines. The primary amine may be an integral part of the signalling moiety or be chemically added. That is, the signalling moiety either contains a primary amine or can be modified to contain a primary amine in such a manner as to avoid eliminating its signalling function; the addition of the primary amine must not destroy the signalling function. As will be appreciated by those in the art and outlined below, this is generally routine. In a preferred embodiment, the signalling moiety is made with a primary amine; alternatively, the primary amine is added, generally through the use of a linker. That is, in some cases, a linker between the signalling moiety and the primary amine is used. This may serve to functionally “isolate” the signalling moiety, or to either add flexibility (for example to facilitate the attachment of the modified nucleoside to a growing nucleic acid). In the case where the signalling moiety comprises one or more primary amines, it may be desirable in some embodiments to block or protect all but one of the primary amines to allow uniform attachment.

In a preferred embodiment, the signalling moiety is functionalized to facilitate derivatization with a linker comprising primary amine. Thus, a wide variety of signalling moieties are either made or are commercially available which contain functional groups, including, but not limited to, isothiocyanate groups, amino groups, oxo groups, thiol groups, carboxyl groups, haloacetyl groups, maleimides, succinimidyl esters, and sulfonyl halides, all of which may be used to attach a primary amine. Using these functional groups, a linker group comprising a primary amine can be attached, generally using functional groups on the primary amine linker. Thus for example, a short alkyl (including substituted alkyl groups and heteroalkyl groups) linker may be generated with a primary amine on one end and a chemical functional group for attachment to the signalling moiety on the other end. Then an additional linker is used for coupling; for example, homo-or hetero-bifunctional linkers as are well known (see 1994 Pierce Chemical Company catalog, technical section on cross-linkers, pages 155-200, incorporated herein by reference).

In a preferred embodiment, the signalling moiety is an electron transfer moiety, that can be used to detect the presence of the nucleic acid as is generally outlined in U.S. Pat. Nos. 5,591,578; 5,824,473; 5,770,369; 5,705,348 and 5,780,234 and PCT 97/20014; all of which are hereby expressly incorporated by reference. By the terms “electron donor moiety”, “electron acceptor moiety”, and “electron transfer moieties” or grammatical equivalents herein refers to molecules capable of electron transfer under certain conditions. It is to be understood that electron donor and acceptor capabilities are relative; that is, a molecule which can lose an electron under certain experimental conditions will be able to accept an electron under different experimental conditions. It is to be understood that the number of possible electron donor moieties and electron acceptor moieties is very large, and that one skilled in the art of electron transfer compounds will be able to utilize a number of compounds in the present invention. Preferred electron transfer moieties include, but are not limited to, transition metal complexes, organic electron transfer moieties, and electrodes.

In a preferred embodiment, the electron transfer moieties are transition metal complexes. Transition metals are those whose atoms have a partial or complete d shell of electrons. Suitable transition metals for use in the invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinium (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinium, cobalt and iron.

The transition metals (or lanthanide metals, as outlined below) are chelated by at least one polydentate ligand that is covalently attached to the nucleoside. In addition, there may be one or more co-ligands. The ligands provide the coordination atoms for the binding of the metal ion. As will be appreciated by those in the art, the number and nature of the co-ligands will depend on the coordination number of the metal ion. Thus, for example, when the metal has a coordination number of six, the polydentate ligand attached to the ribose of the nucleoside provides at least two coordination atoms, and any combination of mono-, di- or polydentate co-ligands provide the remaining coordination atoms.

As will be appreciated in the art, the co-ligands can be the same or different. Suitable ligands fall into two categories: ligands which use nitrogen, oxygen, sulfur, carbon or phosphorus atoms (depending on the metal ion) as the coordination atoms (generally referred to in the literature as sigma (σ) donors) and organometallic ligands such as metallocene ligands (generally referred to in the literature as pi (π) donors, and depicted herein as Lm). Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,1 1-tetra-azacyclotetradecane (abbreviated cyclam) and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp73-98), 21.1 (pp. 813-898) and 21.3 (pp 915-957), all of which are hereby expressly incorporated by reference.

Suitable sigma donating ligands using carbon, oxygen, sulfur and phosphorus are known in the art. For example, suitable sigma carbon donors are found in Cotton and Wilkenson, Advanced Organic Chemistry, 5th Edition, John Wiley & Sons, 1988, hereby incorporated by reference; see page 38, for example. Similarly, suitable oxygen ligands include crown ethers, water and others known in the art. Phosphines and substituted phosphines are also suitable; see page 38 of Cotton and Wilkenson.

The oxygen, sulfur, phosphorus and nitrogen-donating ligands are attached in such a manner as to allow the heteroatoms to serve as coordination atoms.

In a preferred embodiment, organometallic ligands are used. In addition to purely organic compounds for use as redox moieties, and various transition metal coordination complexes with δ-bonded organic ligand with donor atoms as heterocyclic or exocyclic substituents, there is available a wide variety of transition metal organometallic compounds with π-bonded organic ligands (see Advanced Inorganic Chemistry, 5th Ed., Cotton & Wilkinson, John Wiley & Sons, 1988, chapter 26; Organometallics, A Concise Introduction, Elschenbroich et al., 2nd Ed., 1992, VCH; and Comprehensive Organometallic Chemistry II, A Review of the Literature 1982-1994, Abel et al. Ed., Vol. 7, chapters 7, 8, 10 & 11, Pergamon Press, hereby expressly incorporated by reference). Such organometallic ligands include cyclic aromatic compounds such as the cyclopentadienide ion [C5H5(−1)] and various ring substituted and ring fused derivatives, such as the indenylide (−1) ion, that yield a class of bis(cyclopentadieyl)metal compounds, (i.e. the metallocenes); see for example Robins et al., J. Am. Chem. Soc. 104:1882-1893 (1982); and Gassman et al., J. Am. Chem. Soc. 108:4228-4229 (1986), incorporated by reference. Of these, ferrocene [(C5H5)2Fe] and its derivatives are prototypical examples which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to either the ribose ring or the nucleoside base of nucleic acid. Other potentially suitable organometallic ligands include cyclic arenes such as benzene, to yield bis(arene)metal compounds and their ring substituted and ring fused derivatives, of which bis(benzene)chromium is a prototypical example, Other acyclic π-bonded ligands such as the allyl(−1) ion, or butadiene yield potentially suitable organometallic compounds, and all such ligands, in conduction with other π-bonded and δ-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene are derivatized.

As described herein, any combination of ligands may be used.

In addition to transition metal complexes, other organic electron donors and acceptors may be covalently attached to the nucleoside using the methods of the invention. These organic molecules include, but are not limited to, riboflavin, xanthene dyes, azine dyes, acridine orange, N,N′-dimethyl-2,7-diazapyrenium dichloride (DAP²⁺), methylviologen, ethidium bromide, quinones such as N,N′-dimethylanthra(2,1,9-def-6,5,10-d′e′f′)diisoquinoline dichloride (ADIQ²⁺); porphyrins ([meso-tetrakis(N-methyl-x-pyridinium)porphyrin tetrachloride], varlamine blue B hydrochloride, Bindschedler's green; 2,6-dichloroindophenol, 2,6-dibromophenolindophenol; Brilliant crest blue (3-amino-9-dimethyl-amino-10-methylphenoxyazine chloride), methylene blue; Nile blue A (aminoaphthodiethylamino phenoxazine sulfate), indigo-5,5′,7,7′-tetrasulfonic acid, indigo-5,5′,7-trisulfonic acid; phenosafranine, indigo-5-monosulfonic acid; safranine T; bis(dimethylglyoximato)-iron(II) chloride; induline scarlet, neutral red, anthracene, coronene, pyrene, 9-phenylanthracene, rubrene, binaphthyl, DPA, phenothiazene, fluoranthene, phenanthrene, chrysene, 1,8-diphenyl-1,3,5,7-octatetracene, naphthalene, acenaphthalene, perylene, TMPD and analogs and subsitituted derivatives of these compounds.

In a preferred embodiment, the signalling moiety is an optical dye. By “optical dye” herein is meant a photoluminescent compound. That is, a compound that will emit detectable energy after excitation with light. In a preferred embodiment, the optical dye is fluorescent; that is, upon excitation with a particular wavelength, the optical dye with emit light of a different wavelength; such light is typically unpolarized. In an alternative embodiment, the optical dye is phosphorescent.

Preferred optical dyes include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Suitable optical dyes are described in the Sixth Edition of the Molecular Probes Handbook by Richard P. Haugland, hereby expressly incorporated by reference in its entirety; see chapters 1, 2 and 3 in particular. Many fluorophores are commercially available as derivatives that will react with amines (chapter 1), thiols (chapter 2) and other functional groups including alcohols, aldehydes, ketones, carboxylic acids, etc (chapter 3). Also preferred are fluorescent lanthanide metal ion chelates, including, but not limited to, Er, Tb, and Dy, as are known in the art.

As will be appreciated by those in the art, a large number and variety of fluorochromes are known, both that either contain a non-essential primary amine or can be easily derivatized to contain a primary amine.

In a preferred embodiment, the signalling moiety is a dye or chromophore, i.e. a moiety detectable in the visible spectrum.

In a preferred embodiment, the signalling moiety is an MRI contrast agent. By “MRI contrast agent” herein is meant a molecule that can be used to enhance the MRI image. As is known in the art, MRI contrast agents generally comprise a paramagnetic metal ion bound to a chelator. By “paramagnetic metal ion”, “paramagnetic ion” or “metal ion” herein is meant a metal ion which is magnetized parallel or antiparallel to a magnetic field to an extent proportional to the field. Generally, these are metal ions which have unpaired electrons; this is a term understood in the art. Examples of suitable paramagnetic metal ions, include, but are not limited to, gadolinium III (Gd+3 or Gd(III)), iron III (Fe+3 or Fe(III)), manganese II (Mn+2 or Mn(II)), yttrium III (Yt+3 or Yt(III)), dysprosium (Dy+3 or Dy(III)), and chromium (Cr(III) or Cr+3). In a preferred embodiment the paramagnetic ion is the lanthanide atom Gd(III), due to its high magnetic moment (U²=63BM2), a symmetric electronic ground state (S8), and its current approval for diagnostic use in humans.

In addition to the metal ion, the MRI contrast agent usually comprise a chelator. Due to the relatively high toxicity of many of the paramagnetic ions, the ions are rendered nontoxic in physiological systems by binding to a suitable chelator. The chelator utilizes a number of coordination atoms at coordination sites to bind the metal ion. As is discussed below, the replacement of a coordination atom with either a primary amine or a functional group for attachment of a primary amine linker may render the metal ion complex more toxic by decreasing the half-life of dissociation for the metal ion complex. Thus, in a preferred embodiment, a site other than a coordination site is preferably used for covalent attachment to the nucleoside. However, for some applications, e.g. analysis of tissue and the like, the toxicity of the metal ion complexes may not be of paramount importance and thus covalent attachment via a coordination site is appropriate. Similarly, some metal ion complexes are so stable that even the replacement of one or more additional coordination atoms with a blocking moiety does not significantly effect the half-life of dissociation. For example, both DTPA and DOTA, described below, are extremely stable when complexed with Gd(III). Accordingly, one or several of the coordination atoms of the chelator may be replaced with one or more functional moieties for covalent attachment without a significant increase in toxicity.

There are a large number of known macrocyclic chelators or ligands which are used to chelate lanthanide and paramagnetic ions. See for example, Alexander, Chem. Rev. 95:273-342 (1995) and Jackels, Pharm. Med. Imag, Section III, Chap. 20, p645 (1990), expressly incorporated herein by reference, which describes a large number of macrocyclic chelators and their synthesis. Similarly, there are a number of patents which describe suitable chelators for use in the invention, including U.S. Pat. Nos. 5,155,215, 5,087,440, 5,219,553, 5,188,816, 4,885,363, 5,358,704, 5,262,532, and Meyer et al., Invest. Radiol. 25: S53 (1990), all of which are also expressly incorportated by reference. There are a variety of factors which influence the choice and stability of the chelate metal ion complex, including enthalpy and entropy effects (e.g. number, charge and basicity of coordinating groups, ligand field and conformational effects, etc.). In general, the chelator has a number of coordination atoms which are capable of binding the metal ion. The number of coordination atoms, and thus the structure of the chelator, depends on the metal ion. Thus, as will be understood by those in the art, any of the known paramagnetic metal ion chelators or lanthanide chelators can be easily modified using the teachings herein to add a primary amine as outlined herein for attachment to a nucleoside.

Preferred MRI contrast agents include, but are not limited to, 1,4,7,10-tetraazacyclododecane-N,N′, N″, N′″-tetracetic acid (DOTA), diethylenetriaminepentaacetic (DTPA), 1,4,7,10-tetraazacyclododecane-N,N′,N″,N′″-tetraethylphosphorus (DOTEP), 1,4,7,10-tetraazacyclododecane-N,N′,N″-triacetic acid (Do3A) and derivatives thereof (see U.S. Pat. Nos. 5,188,816, 5,358,704, 4,885,363, and 5,219,553, hereby expressly incorporated by reference).

As is described herein, in a preferred embodiment the MRI agent is substituted at any number of possible positions with either a primary amine or a functional group to facilitate the addition of the primary amine for covalent attachment to the nucleoside. For example, when the contrast agent is DOTA, a preferred embodiment utilizes any one of the R sites of structure 1 as the site of attachmen of a primary amine.

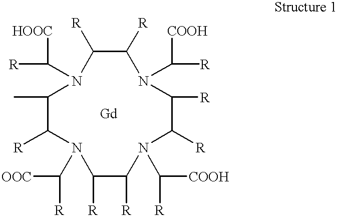

In an additional embodiment, one of the carboxylic acid chelating “arms” (i.e. a coordination atom) of DOTA may also be used as the site of covalent attachment, as depicted in Structure 2 (unsubstituted DOTA, although substituted compounds as also included):

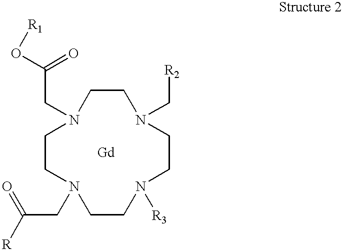

In this embodiment, either a carbonyl (i.e. as depicted with R in Structure 2), an ester linkage (i.e. as depicted with R₁), or direct linkage to the nitrogen atom (as depicted in R₂ and R₃) may be used as the site of attachment of a primary amine-containing moiety, depending on the type of coupling chemistry used. As will be appreciated by those in the art, similar substitutions may be done with DTPA and DOTEP (the unsubstituted forms of which are depicted in Structures 3 and 4, respectively, although as outlined above in Structure 1, the carbon atoms of DTPA and DOTEP or any other MRI agent of use herein may be substituted with R groups, at least one of which contains a primary amine):

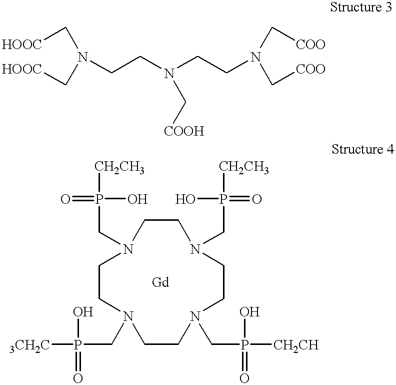

Other suitable Gd(III) chelators are described in Alexander, supra, Jackels, supra, Lauffer et al., supra, U.S. Pat. Nos. 5,155,215, 5,087,440, 5,219,553, 5,188,816, 4,885,363, 5,358,704, 5,262,532, and Meyer et al., Invest. Radiol. 25: S53 (1990), among others.

As will be appreciated by those in the art, the R sites outlined herein may also comprise additional substitution groups, in addition to the R site that ultimately comprises a primary amine. Suitable substitution groups include a wide variety of groups, as will be understood by those in the art. For example, suitable substitution groups include substitution groups disclosed for DOTA and DOTA-type compounds in U.S. Pat. Nos. 5,262,532, 4,885,363, amd 5,358,704. These groups include hydrogen, alkyl (including substituted alkyl groups and heteroalkyl groups), aryl groups (including substituted aryl and heteroaryl groups), alcohol, amino, amido, nitro, ethers, esters, aldehydes, sulfonyl, silicon moieties, halogens, sulfur containing moieties, phosphorus containing moieties, and ethylene glycols, etc. Preferred substitution groups include hydrogen. As will be appreciated by those skilled in the art, each position outlined herein may have two R groups attached (R′ and R″), although in a preferred embodiment only a single R group is attached at any particular position. Thus, for example, the MRI contrast agents utilized in the invention may be substituted at any one of the R positions with moieties to confer or neutralize charge, alter the hydrophobicity or hydrophilicity, or alter the molecular weight. The larger the molecule, the slower it rotates in solution and the relaxivity increases.

Chelators for use with other metals are known. For example, suitable chelators for Fe(III) ions are well known in the art. See Lauffer et al., J. Am. Chem. Soc. 109:1622 (1987); Lauffer, Chem. Rev. 87:901-927 (1987); and U.S. Pat. Nos. 4,885,363, 5,358,704, and 5,262,532, all which describe chelators suitable for Fe(III). Suitable chelators for Mn(II) ions are also well known in the art; see for example Lauffer, supra, and U.S. Pat. Nos. 4,885,363, 5,358,704, and 5,262,532. Suitable chelators for Yt(III) ions include, but are not limited to, DOTA and DPTA and derivatives thereof (see Moi et al., J. Am. Chem. Soc. 110:6266-6267 (1988)) and those chelators described in U.S. Pat. No. 4,885,363 and others, as outlined above. Chelators for Dy+3 (Dy(III)) are also known in the art and described in the references cited herein.

In a preferred embodiment, the signalling moiety is a chemiluminescent or electrochemiluminescent label such as Ru-tris-bipyridyl and others outlined in W090/05301, WO 92/14139, and U.S. Pat. Nos. 5,779,976 and 5,770,459, and related materials.

In a preferred embodiment, the signalling moiety is a binding ligand of a binding pair, such that a secondary binding event binds the label to the nucleoside or nucleic acid comprising the nucleoside. In this embodiment, suitable binding partner pairs include haptens-antibodies, biotin-streptavidin, etc. Similarly, the signalling moiety is an enzyme that generates a label or a change in a label upon catalysis, although in some embodiments this is not preferred.

In some embodiments, as will be appreciated by those in the art, the synthesis of an intermediate or precursor to the signalling moiety is made; that is, a polydentate ligand that forms the core of the signalling moiety is added using the methods of the invention, and then the metal ion is added later. For example, as shown in the Figures, a bidentate ligand for the subsequent attachment of ruthenium can be added to the nucleoside, followed by incorporation of the co-ligands and the metal ion. Alternatively, nucleosides modified with DOTA can be made, with subsequent attachment of the gadolinium ion. Since detection of the signalling moiety relies on the presence of the metal ion in both of these instances, the methods of the invention generate a nucleoside modified with a polydentate ligand as an “intermediate” or “signalling moiety precursor” rather than the completed signalling moiety. Accordingly, nucleosides with polydentate ligands may be made.

Accordingly, the invention provides methods for making modified nucleosides comprising a covalently attached signalling moiety or signalling moiety precursor. By “covalently attached” herein is meant that two moieties are attached by at least one bond, including sigma bonds, pi bonds and coordination bonds. The signalling moiety is covalently attached to either the 2′ or 3′ position of the ribose or to an equivalent position in a nucleoside analog.

The methods comprising adding an anhydro-nucleoside and a signalling moiety or precursor comprising a primary amine. By “anhydro-nucleoside” herein is meant a 2,2′-, 2,3′- or 2,5′ anhydronucleoside, comprising an oxygen bridge between the C-2 of the base pyrimidine and the C-2′ or C-3′ of the ribose or ribose analog. See for example Sebesta et al., Tetrahedron 52:14385 (1996); McGee et al., J. Org. Chem. 61:779 (1996); Fox et al., Advances in Carbohydrate Chemistry 14:283 (1959); Codington et al., J. Org. Chem. Jan. 1962, pages 163-167; Glinski et al., J. Org. Chem. 38:4299 (1973); Kirschenheuter et al., Tetrahedron Lett. 35:8517 (1994); Mengel et al., Angew. Chem. Int. Ed. Engl. 17:525 (1978); Miller et al., 29:1772 (1964); and references cited therein, all of which are expressly incorporated by reference. In some cases, as is generally outlined in Moffatt, Transformations of the Sugar Moiety of Nucleosides, pp7l-164, hereby expressly incorporated by reference, anhydronucleosides of purines can be made, forming a bridge between the N-3 of the purine and the 2′,3′ or 5′ position on the ribose. As will be appreciated by those in the art, anhydronucleosides can be made with all the bases, and many base analogs. This is done as is well known in the art; see Moffatt, supra, Sebesta, supra, and McGee, supra.

For the attachment of the signalling moiety to the 2′ position of the nucleoside, a 2,2′ anhydronucleoside is used (using pyrimidine numbering; purines will use the 4,2′,4,3′ and 4,5′ positions to the N-3 nitrogen). For attachment to the 3′ position, a 2,3′ anhydronucleoside is used. For attachment to the 5′ position, a 2,5′ anhydronucleoside is made.

In addition, any free hydroxyl groups on the nucleoside are preferably protected with a protecting group to prevent reactions at this site (i.e. generally at the 5′ position when the signalling moiety is added to either the 2′ or 3′ position); suitable protecting groups are known in the art and include MMT, DMT, and silyl containing protecting groups; see Greene, Protecting Groups in Organic Synthesis, 2d Ed., John Wiley & Sons, 1991, hereby incorporated by reference.

The anhydro-nucleoside and the signalling moiety (or precursor) comprising a primary amine are added together in the presence of an activation agent to form an activated anhydronucleoside. By “activation agent” herein is meant an agent that activates the carbon for attack by the nucleophile, i.e. the nitrogen of the primary amine; that is, the agent allows the formation of carbamates. Suitable activation agents, include, but are not limited to, carbonyldimidazole, m-nitrophyenylchloroformate and p-nitrophyenylchloroformate. The anhydro-nucleoside and the signalling moiety comprising a primary amine are added generally in a 1:1 ratio. The activation agent will be added at concentrations known in the art, see Sebesta et al. supra, and McGee, supra.

By “activated anhydronucleoside” herein is meant the anhydronucleoside ready to react with the signalling moiety comprising a primary amine to form a carbamate.

The activated anhydronucleoside is then treated with a cyclization agent to form a cyclized intermediate. By “cyclization agent” herein is meant an agent such as a weak base that breaks the 2,2′-, 2,3′- or 2,5′ oxygen bridge between the ribose and the base and forms a ring structure including the 2′ and 3′ positions of the ribose, such that at either the 2′ or 3′ position a nitrogen atom is directly attached. Suitable cyclization agents include, but are not limited to, 1,8 diazabicyclo-undec-7-ene (DBU) and 1,4-diazabicyclo-octane (DBO).

The cyclized intermediate is then treated with a strong base such as NaOH to form the modified nucleoside.

As will be appreciated by those in the art, purification steps may be included after each step, if desired or required.

Once formed, the modified nucleosides may be treated in a number of ways. In a preferred embodiment, the signalling moiety is attached at the 2′ position of the ribose, and the nucleoside is then protected and further modified to be incorporated into a growing oligonucleotide by standard synthetic techniques (Gait, Oligonucleotide Synthesis: A Practical Approach, IRL Press, Oxford, UK 1984; Eckstein) in several ways. In one embodiment, one or more modified nucleosides are converted to the triphosphate form and incorporated into a growing oligonucleotide chain by using standard molecular biology techniques such as with the use of the enzyme DNA polymerase I, T4 DNA polymerase, T7 DNA polymerase, Taq DNA polymerase, reverse transcriptase, and RNA polymerases. This is generally done as long as the modified nucleoside is soluble. For the incorporation of a 3′ modified nucleoside to a nucleic acid, terminal deoxynucleotidyltransferase may be used. (Ratliff, Terminal deoxynucleotidyltransferase. In The Enzymes, Vol 14A. P. D. Boyer ed. pp 105-118. Academic Press, San Diego, Calif. 1981). Alternatively, and preferably, the amino nucleoside is converted to the phosphoramidite or H-phosphonate form, which are then used in solid-phase or solution syntheses of oligonucleotides. In this way the modified nucleoside is incorporated into the oligonucleotide at either an internal position or a terminus. This is generally done in one of two ways. First, the 5′ position of the ribose is protected with 4′,4-dimethoxytrityl (DMT) followed by reaction with either 2-cyanoethoxy-bis-diisopropylaminophosphine in the presence of diisopropylammonium tetrazolide, or by reaction with chlorodiisopropylamino 2′-cyanoethyoxyphosphine, to give the phosphoramidite as is known in the art; although other techniques may be used as will be appreciated by those in the art. See Gait, supra; Caruthers, Science 230:281 (1985), both of which are expressly incorporated herein by reference.

For attachment of a signalling moiety to the 3′ terminus of a nucleic acid, a preferred method utilizes the attachment of the modified nucleoside to controlled pore glass (CPG) or other oligomeric supports. In this embodiment, the modified nucleoside is protected at the 5′ end with DMT, and then reacted with succinic anhydride with activation. The resulting succinyl compound is attached to CPG or other oligomeric supports as is known in the art. Further phosphoramidite nucleosides are added, either modified or not, to the 5′ end after deprotection.

When a metal ion complex is a component of the signalling moiety, synthesis may occur in several ways. In a preferred embodiment, the ligand(s) are added to a nucleoside, followed by the metal ion, and then the nucleoside with the metal ion complex attached is added to an oligonucleotide, i.e. by addition to the nucleic acid synthesizer. Alternatively, the ligand(s) may be attached, followed by incorportation into a growing oligonucleotide chain, followed by the addition of the metal ion.

In a preferred embodiment, a single modified nucleoside comprising a signalling moiety is incorporated into a single oligonucleotide. In a firther preferred embodiment, a plurality of modified nucleosides are incorporated into the oligonucleotide.

Thus, the present invention provides methods to make nucleic acids comprising at least one signalling moiety. The nucleic acids of the invention find use in a wide variety of applications.

In a preferred embodiment, the compositions of the invention are used to detect target sequences in a sample. The term “target sequence” or grammatical equivalents herein means a nucleic acid sequence on a single strand of nucleic acid. The target sequence may be a portion of a gene, a regulatory sequence, genomic DNA, cDNA, RNA including mRNA and rRNA, or others. It may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. As is outlined more fully below, probes are made to hybridize to target sequences to determine the presence or absence of the target sequence in a sample. Generally speaking, this term will be understood by those skilled in the art. The target sequence may also be comprised of different target domains; for example, a first target domain of the sample target sequence may hybridize to a capture probe on a surface and a second target domain may hybridize to a portion of reporter probe containing a signalling moiety, etc. The target domains may be adjacent or separated. The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain. That is, the nucleic acids of the invention may find particular use in sandwich hybridization assays, where multiple probes are used.

If required, the target sequence is prepared using known techniques. For example, the sample may be treated to lyse the cells, using known lysis buffers, electroporation, etc., with purification and/or amplification such as PCR occuring as needed, as will be appreciated by those in the art.

Probes of the present invention are designed to be complementary to a target sequence (either the target sequence of the sample or to other probe sequences, as is described herein) such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

Generally, the nucleic acid compositions made by the methods of the invention are useful as oligonucleotide probes. As is appreciated by those in the art, the length of the probe will vary with the length of the target sequence and the hybridization and wash conditions. Generally, oligonucleotide probes range from about 8 to about 50 nucleotides, with from about 10 to about 30 being preferred and from about 12 to about 25 being especially preferred. In some cases, very long probes may be used, e.g. 50 to 200-300 nucleotides in length. Thus, in the structures depicted herein, nucleosides may be replaced with nucleic acids.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by referenece. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Thus, the compositions made by the methods of the present invention may be used in a variety of research, clinical, quality control, or field testing settings.

In a preferred embodiment, the probes are used in genetic diagnosis. For example, probes can be made using the techniques disclosed herein to detect target sequences such as the gene for nonpolyposis colon cancer, the BRCA1 breast cancer gene, P53, which is a gene associated with a variety of cancers, the Apo E4 gene that indicates a greater risk of Alzheimer's disease, allowing for easy presymptomatic screening of patients, mutations in the cystic fibrosis gene, or any of the others well known in the art.

In an additional embodiment, viral and bacterial detection is done using the complexes of the invention. In this embodiment, probes are designed to detect target sequences from a variety of bacteria and viruses. For example, current blood-screening techniques rely on the detection of anti-HIV antibodies. The methods disclosed herein allow for direct screening of clinical samples to detect HIV nucleic acid sequences, particularly highly conserved HIV sequences. In addition, this allows direct monitoring of circulating virus within a patient as an improved method of assessing the efficacy of anti-viral therapies. Similarly, viruses associated with leukemia, HTLV-I and HTLV-II, may be detected in this way. Bacterial infections such as tuberculosis, clymidia and other sexually transmitted diseases, may also be detected.

In a preferred embodiment, the nucleic acids of the invention find use as probes for toxic bacteria in the screening of water and food samples. For example, samples may be treated to lyse the bacteria to release its nucleic acid, and then probes designed to recognize bacterial strains, including, but not limited to, such pathogenic strains as, Salmonella, Campylobacter, Vibrio cholerae, Leishmania, enterotoxic strains of E. coli, and Legionnaire's disease bacteria. Similarly, bioremediation strategies may be evaluated using the compositions of the invention.

In a further embodiment, the probes are used for forensic “DNA fingerprinting” to match crime-scene DNA against samples taken from victims and suspects.

In an additional embodiment, the probes in an array are used for sequencing by hybridization. In a preferred emboidment, PCR primers are made comprising signalling moieties, and detection of target sequences utilizes the incorporation of the labelled PCR products.

Thus, the present invention provides for extremely specific and sensitive probes, which may, in some embodiments, detect target sequences without removal of unhybridized probe. This will be useful in the generation of automated gene probe assays.

Labelled nucleic acids made using the methods of the invention can be can be attached to solid supports as is well known in the art.

When the signalling moiety is an MRI contrast agent, the nucleic acids can be used in a variety of imaging modalities; see PCT U.S. Pat. No. 95/14621, hereby incorporated by reference.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLE

### Syntheis of Polydentate Nucleoside

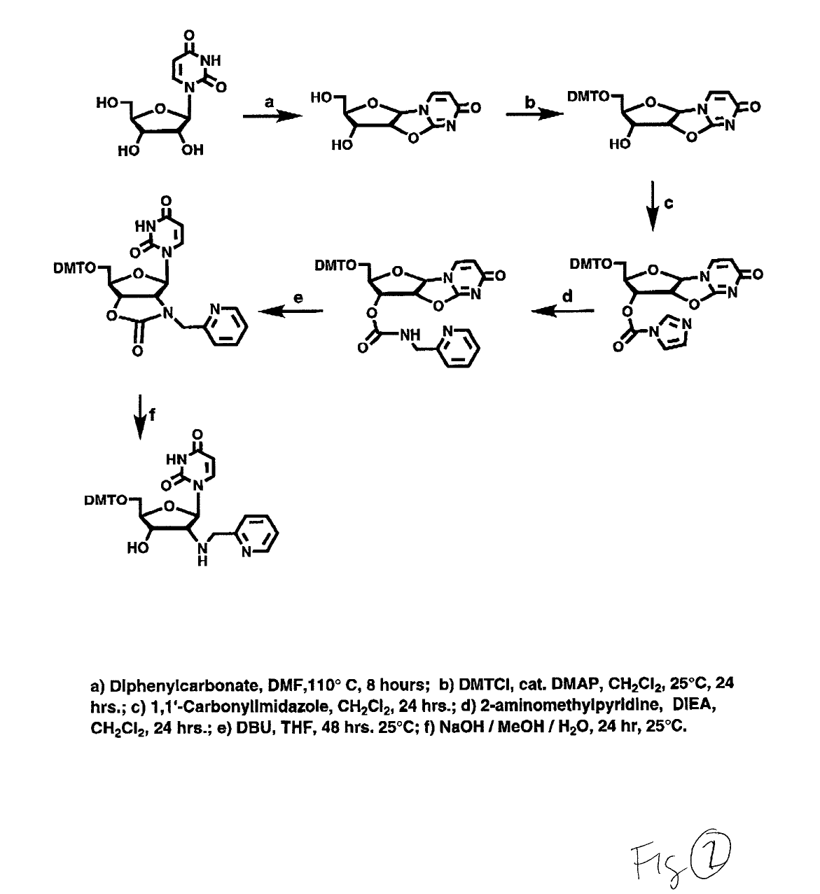

This example is directed to the synthesis shown in FIG. 2. 2-amino-ethylpyridine may be subsituted as well.

To uridine that was slurried in DMF was added diphenylcarbonate and heated to 110° C. for 8 hours. The product was purified by flash column chromatography. The cyclized intermediate was dissolved in CH₂Cl₂ and a catalytic amount of DMAP added along with a 1.1 excess of DMTC1 and kept at room temperature for 24 hours. The solution was evaporated to dryness and purified by flash column chromatography. The purified product was dissolved in CH2Cl2 and 2 equivalents of 1,1′-carbonyldimidazole added and allowed to react for 24 hrs. To this solution was added 1 equiv of DIEA and 1.1 equiv. of 2-aminomethylpyridine and allowed to react for and additional 24 hours.

After purification (not necessary) the product was suspended in THF and DBU added and allowed to react for 48 hours. This material was treated with NaOH in methanol/water for 24 hours at room temperature. The product was purified by flash column chromatograpy and characterized. 1HNMR and mass spec. confirmed the expected product.

We claim:

1. A method for making a modified nucleoside comprising a covalently attached signalling moiety or signalling moiety precursor, said method comprising:
a) adding an anhydro-nucleoside and a signalling moiety or signalling moiety precursor comprising a primary amine in the presence of an activation agent to form an activated anhydro-nucleoside;
b) treating said activated anhydro-nucleoside with a cyclization agent to form a cyclized intermediate; and
c) treating said cyclized intermediate with a base to form said modified nucleoside.

2. A method according to claim 1 further comprising adding a phosphoramidite group to said modified nucleoside.

3. A method according to claim 2 further comprising incorporating said phosphoramidite modified nucleoside into a growing nucleic acid.

4. A method according to claim 1 wherein said nucleoside is a naturally occuring nucleoside.

5. A method according to claim 1 wherein said nucleoside is a nucleoside analog.

6. A method according to claim 1 wherein said activating agent is carbonyldimidazole.
